(19) United States
(12) Patent Application Publication
(10) Pub. No.: US 2022/0096587 A1
(43) Pub. Date: Mar. 31, 2022

(54) Compositions and Methods for Treating and Preventing Helicobacter Pylori Infections

(71) Applicant: Locus IP Company, LLC, Solon, OH (US)

(72) Inventors: Sean FARMER, Ft. Lauderdale, FL (US); Ken ALIBEK, Solon, OH (US); Albina TSKHAY, Solon, OH (US)

(21) Appl. No.: 17/296,326

(22) PCT Filed: Nov. 26, 2019
(86) PCT No.: PCT/US2019/063262

§ 371 Date: May 24, 2021

Related U.S. Application Data
(60) Provisional application No. 62/772,260, filed on Nov. 28, 2018.

(51) Int. Cl. A61K 36/899; A61K 36/8962; A61K 36/534; A61K 36/9068; A61K 31/4439; A61K 9/127; A61K 45/06; A61K 47/54

(52) U.S. Cl. CPC A61K 36/899; A61K 36/8962; A61K 36/534; A61K 47/543; A61K 31/4439; A61K 9/127; A61K 45/06; A61K 36/9068

(57) ABSTRACT

The present invention provides compositions and methods for treating subject infected with Helicobacter pylori. The invention utilizes natural supplement compositions comprising a blend of essential oils, as well as proton-pump inhibitors and biosurfactants. Advantageously, the present invention can lead to simultaneous improvement of diseases, disorders and conditions caused by H.pylori infection, reduction in the occurrence of H. pylori infections, and reduction in the development of antibiotic-resistant strains of the bacteria.

### CROSS-REFERENCE TO RELATED APPLICATION

This application claims priority to U.S. Provisional Patent Application No. 62/772,260, filed Nov. 28, 2018, which is incorporated herein by reference in its entirety.

### BACKGROUND OF INVENTION

Helicobacter pylori is one of the world's most prevalent bacterial infections. This pathogen is commonly transmitted between people via saliva, but can also be spread by fecal contamination of food or water. In places where water treatment is undeveloped, conditions are crowded, and hygiene and sanitation are poor, H. pylori prevalence is even higher.

H. pylori is a spiral-shaped bacterium that infects the stomach or first part of the small intestine, where it burrows into the stomach or intestinal lining and causes inflammation (gastritis). H. pylori can form biofilms, which helps it survive in the harsh acidic environment of the stomach. Furthermore, it can produce the enzyme urease, which neutralizes the acid by reacting with urea to form ammonia.

An infection by H. pylori causes upper digestive tract disorders and complications, including chronic gastritis, ulcers, life-threatening bleeding, non-ulcer dyspepsia, and mucosa-associated lymphoid tissue (MALT) lymphoma. H. pylori may even cause food allergies in children (see Corrado et al. 1998). Furthermore, approximately 75% of cases of gastric cancers in developed countries, and more than 90% in developing countries, are caused by H. pylori.

In addition to causing severe disorders of the gastrointestinal (GI) tract, this pathogen has also been indicated as playing a role in several non-GI tract-related health conditions.) These include many cancers, migraines (see Hosseinzadeh et al. 2011; Ansari et al. 2015), cardiovascular and cerebrovascular diseases, liver and pancreatic diseases, such as hepatocellular carcinoma, cirrhosis and hepatic encephalopathy, nonalcoholic fatty liver disease and fibrosis, acute and chronic pancreatitis pathogenesis, autoimmune pancreatitis, diabetes mellitus and metabolic syndrome. Furthermore, infection with H. pylori may lead to enhanced cognitive impairment in certain neurodegenerative conditions, such as Alzheimer's and Parkinson's diseases (see Raubod-Caudron et al. 2012).

Currently, the most common treatment for H. pylori infection is the administration of antibiotics and proton-pump inhibiting medications. Typically, the first-line therapy consists of two antibiotics and one proton-pump inhibitor: clarithromycin, amoxicillin and omeprazole. Such an approach has limitations, however. There is a high likelihood of recurrence due to the ease with which the infection spreads. Thus, antibiotics are only a temporary solution. Second, H. pylori has developed a resistance to many antibiotics that were once effective.

Another approach that has been posed for reducing the numbers of H. pylori infections is the administration of an H. pylori vaccine, particularly in developing countries. However, the unique character and persistence of H. pylori have created obstacles preventing the development of an effective vaccine to date.

Thus, given the prevalence of H. pylori infections and the many associated diseases and conditions thereto, there is a need for effective compositions and methods for treating and preventing H. pylori infections.

### BRIEF SUMMARY

The present invention provides compositions and methods for treating and/or preventing H. pylori infections, as well as for treating and/or preventing the development of symptoms, comorbidities, and diseases associated with H. pylori infections. Embodiments of the present invention provide naturally-derived compositions and methods for controlling H. pylori infecting a subject's digestive tract. Advantageously, in one embodiment, the present invention can be useful against antibiotic-resistant H. pylori.

In certain embodiments, the present invention provides a supplement composition for treating and/or preventing an H. pylori infection, wherein the composition comprises ingredients that help support immune health and suppress infectious agents in the subject's body. Additionally, the composition can comprise ingredients that are considered natural or naturally-derived.

In one embodiment, the supplement composition comprises therapeutically-effective amounts of essential oils selected from lemongrass oil, garlic oil, oregano oil, Satureja bachtiarica oil and ginger oil.

In one embodiment the supplement composition comprises, consists of, or consists essentially of therapeutically-effective amounts of lemongrass oil, garlic oil, oregano oil, Satureja bachtiarica oil and ginger oil.

In certain embodiments, the supplement composition further comprises a therapeutically-effective amount of a proton-pump inhibitor, such as, for example, omeprazole. Advantageously, the addition of the proton-pump inhibitor can help overcome the protection provided H. pylori's ability to form biofilms.

In certain embodiments, the supplement composition further comprises a biological amphiphilic molecule. In a specific embodiment, the biological amphiphilic molecule is a surfactant, preferably a biosurfactant. Surfactants are capable of reducing surface tension, among other capabilities, and thus provide additional control mechanisms against H. pylori.

In one embodiment, the composition comprises a glycolipid biosurfactant. In a specific embodiment, the glycolipid is a sophorolipid (SLP). SLP has the ability to destroy biofilms, and furthermore, has anti-inflammatory, tissue-healing, antibacterial and antioxidant properties.

In one embodiment, the components of the supplement composition are formulated as a mixture, comprising optional additional ingredients, such as, for example, a pharmaceutically-acceptable carrier.

In one embodiment, the supplement composition is formulated into a biosurfactant delivery system, wherein a biosurfactant forms a liposome, or a micro- or nanocapsule, with the supplement composition encapsulated therein. In one embodiment, additional biological polymers can be included to provide further structure for encapsulation.

Encapsulating with biosurfactants can enhance the bioavailability of the supplemental compound by protecting the compound from components in the blood, such as proteins and other molecules, that otherwise might bind to the compound and prevent it from penetrating a target site. Additionally, the encapsulated delivery system can allow for antibacterial compounds that might otherwise be degraded by acids or enzymes in the GI tract to be administered orally, as it creates a barrier against the acids or enzymes. Furthermore, the encapsulated delivery system formulation allows for time release of the antibacterial compound, thereby reducing the potential toxicity or potential negative side-effects of a compound in a subject.

In preferred embodiments, the present invention provides methods for treating and/or preventing a Helicobacter infection in a subject, wherein the method comprises administering to the subject a therapeutically-effective amount of a supplement composition of the present invention.

In one embodiment, the method can be used to treat symptoms of H. pylori infection, including GI symptoms and non-digestive symptoms.

The subject can be any mammal who is infected with H. pylori or who is at risk of becoming infected therewith. In preferred embodiments, the subject is a human.

In one embodiment, the method first comprises testing the subject for, and/or diagnosing the subject with, H. pylori infection. In one embodiment, the testing is performed using known testing methods, including blood antibody test, urea breath test, stool antigen test, and stomach biopsy.

Advantageously, the present invention can lead to improvement of diseases, disorders and conditions caused by H. pylori infection, reduction in the occurrence of H. pylori infections, and reduction in the development of antibiotic-resistant strains of the bacteria.

### DETAILED DISCLOSURE

The present invention provides compositions and methods for treating and/preventing Helicobacter pylori infections, as well as the diseases, disorders and conditions associated with such an infection. Advantageously, the present invention can utilize naturally-derived substances to control H. pylori in the subject, including strains that have become resistant to antibiotics.

### Selected Definitions

As used herein, the term “subject” refers to an animal who has been infected by Helicobacter or who is at risk of being infected therewith. The animal may be selected from, for example, pigs, horses, goats, cats, mice, rats, dogs, primates, e.g., apes, chimpanzees and orangutans, guinea pigs, hamsters, cows, sheep, birds, e.g., chickens, reptiles, fish, as well as any other vertebrate or invertebrate. The preferred subject in the context of this invention is a mammal. Even more preferably, the subject is a human of any gender and any age or stage of development, including infant, toddler, adolescent, teenager, adult, middle-aged and senior.

As used herein, “infection” refers to the introduction and/or presence of a disease-causing, or pathogenic, organism into and/or in another organism, tissue or cell.

As used herein, “treating” or “treatment” means the eradicating, improving, reducing, ameliorating or reversing of at least one sign or symptom of a disease, condition or disorder (e.g., an infection). Treatment can include, but does not require, a complete cure of the disease, condition or disorder, meaning treatment can also include partial eradication, improvement, reduction, amelioration or reversal.

As used herein “preventing” or “prevention” of a disease, condition or disorder means delaying, inhibiting, suppressing, forestalling, and/or minimizing the onset or progression of a particular sign or symptom thereof. Prevention can include, but does not require, indefinite, absolute or complete prevention throughout a subject's lifetime, meaning the sign or symptom may still develop at a later time and/or with a lesser severity than it would without preventative measures. Prevention can include reducing the severity of the onset of such a disease, condition or disorder, and/or inhibiting the progression of the condition or disorder to a more severe condition or disorder.

As used herein, “control” in the context of a microorganism refers to killing and/or eradicating a microbial species, or otherwise reducing the population numbers and/or inhibiting pathogenicity of further growth of the microbial species at a particular site. In one embodiment, when the microorganism has caused an infection, controlling the microorganism can be a form of treating the infection.

The terms “therapeutically effective” amount or dose, “effective amount,” and “effective dose” are used in this disclosure to refer to an amount of a compound or composition that, when administered to a subject, is capable of providing a desired therapeutic effect (e.g., treatment of an infection) or a desired level of treatment. The actual amount of the compound or composition will vary depending on a number of factors including, but not limited to, the particular disease being treated, the severity of the disease, the size and health of the patient, and the route of administration. A skilled medical practitioner having the benefit of the subject disclosure can readily determine the appropriate amount using methods known in the medical arts.

A plant “extract,” as used herein, refers to the material resulting from exposing a plant part to a solvent and removing the solvent, or from using various chemical, immunological, biochemical or physical procedures known to those of skill in the art, including but not limited to, precipitation, steam distillation, centrifugation, filtering, column chromatography, detergent lysis and cold pressing (or expression). Plant extracts can include, for example, essential oils. Plant material can include roots, stems, leaves, flowers, or parts thereof.

As used herein, the term “probiotic” refers to microorganisms, which, when administered in adequate amounts, confer a health benefit on the host. In certain embodiments, probiotics are administered to a subject's digestive tract to confer, for example, digestive benefits to the subject.

The terms “natural” and “naturally-derived,” as used in the context of a chemical compound or substance is a material that is found in nature, meaning that it is produced from earth processes or by a living organism. A natural product can also be isolated or purified from its natural source of origin and utilized in, or incorporated into, a variety of applications, including foods, beverages, cosmetics, and supplements. Thus, natural products can be combined with other natural or non-natural products, with which they are not found in nature. A natural product can also be produced in a lab by chemical synthesis, provided no artificial components or ingredients (i.e., synthetic ingredients that cannot be found naturally as a product of the earth or a living organism) are added.

The terms “isolated” or “purified,” when used in connection with biological or natural materials such as nucleic acid molecules, polynucleotides, polypeptides, proteins, organic compounds, such as small molecules, microorganism cells/strains, or host cells, means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

The description herein of any aspect or embodiment of the invention using terms such as “comprising,” “having,” “including,” or “containing” with reference to an element or elements is intended to provide support for a similar aspect or embodiment of the invention that “consists of,” “consists essentially of,” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The term “consisting essentially of,” as used herein, limits the scope of the ingredients and steps to the specified materials or steps and those that do not materially affect the basic and novel characteristic(s) of the present invention.

Use of the term “comprising” contemplates other embodiments that “consist” or “consist essentially” of the recited component(s).

### Supplement Compositions

In certain embodiments, the present invention provides a supplement composition for treating and/or preventing an H. pylori infection, wherein the composition comprises, for example, essential oils, as well as other ingredients that help suppress infectious agents in the subject's body. Advantageously, the composition can comprise ingredients that are considered naturally-derived.

In one embodiment, the supplement composition controls Helicobacter spp. microbes that have infected a subject's oral and/or nasal cavities, gallbladder, ears, skin, eyes, stomach, intestine, biliary tract, appendix, and/or other tissues, organs, or systems.

In one embodiment, the supplement composition comprises therapeutically-effective amounts of essential oils selected from lemongrass oil, garlic oil, oregano oil, Satureja bachtiarica oil and ginger oil. In one embodiment, the essential oils are in the form of solutions, wherein the essential oil has been dissolved in an aqueous solvent, such as, e.g., distilled water.

Essential oils, such as those utilized according to the present invention, can have a wide-range of health benefits when administered to a subject. The essential oils according to the present invention, for example, can have antibacterial effects against Helicobater spp. bacteria, most importantly, H. pylori. In some embodiments, the antibacterial effects are strong enough to cause complete eradication of these bacteria. In some embodiments, the essential oils have additional properties that are beneficial for reducing the symptoms of H. pylori infection.

In one embodiment, the supplement composition further comprises a proton-pump inhibitor, such as, e.g., omeprazole.

In one embodiment, the supplement composition further comprises a biological amphiphilic molecule, such as, e.g., a sophorolipid.

In one embodiment the supplement composition comprises, consists of, or consists essentially of therapeutically-effective amounts of an essential oil blend of lemongrass oil, garlic oil, oregano oil, Satureja bachtiarica oil and ginger oil.

In one embodiment, the supplement composition comprises, consists of, or consists essentially of therapeutically-effective amounts of an essential oil blend of lemongrass oil, garlic oil, oregano oil, Satureja bachtiarica oil, ginger oil; a proton-pump inhibitor; and a biological amphiphilic molecule.

In one embodiment, the composition comprises lemongrass oil at a concentration ranging from about 0.001% to about 5.0% [v/v], preferably from about 0.01% to about 2.5%, more preferably about 0.1% to about 1.0%.

In certain embodiments, the lemongrass oil is used as a solution dissolved in distilled water with about 0.1% to about 0.5% [v/v] DMSO, preferably 0.2% DMSO.

In one embodiment, the composition comprises garlic oil at a concentration ranging from about 1 to about 300 ng/ml, preferably about 4 ng/ml to about 260 ng/ml, more preferably about 8μg/ml to about 150 ng/ml.

In one embodiment, the composition comprises oregano oil at a concentration ranging from about 1.5mg/ml to about 2.0 mg/ml. In one embodiment, the composition comprises Satureja bachtiarica oil at a concentration ranging from about 1.5 mg/ml to about 2.0 mg/ml.

In one embodiment, the composition comprises a ratio of oregano oil to S. bachtiarica oil of about 1:1 to about 1:5, preferably about 1:2. The lipophilic structure of these oils enables them to interact with the phospholipid bilayer of the bacterial cell membrane, increasing its permeability and causing cell contents to be released.

In one embodiment, the supplement composition comprises ginger oil at a concentration ranging from about 5.0 μg/ml to about 65 μg/ml, preferably about 6.0 μg/ml to about 55 μg/ml, more preferably about 6.25 μm/ml to about 50 μg/ml.

In addition to antibacterial effects, ginger oil also has anti-oxidant and anti-inflammatory properties and inhibits NF-KB and inflammatory cytokines such as IL-, IL-8 and IL1-beta, which are produced as a result of H. pylori infection. Moreover, it inhibits the production of acid by blocking H-K ATPase, thus supporting the recovery from gastritis and ulcers.

In certain embodiments, the supplement composition further comprises a therapeutically-effective amount of a proton-pump inhibitor (PPI). PPIs work by reducing the amount of stomach acid produced by the glands in the lining of the stomach.

In one embodiment, the PPI enhances the potency of the antibacterial components of the present supplement composition by reducing the amount of stomach acid in the subject's stomach. In one embodiment, the PPI can inhibit urease. In one embodiment, the PPI can have anti-biofilm effects.

In one embodiment, the PPI is a pharmaceutical selected from omeprazole, lansoprazole, dexlansoprazole, esomeprazole, pantoprazole, rabeprazole, ilaprazole and tenatoprazole.

In preferred embodiments, the PPI is omeprazole. Omeprazole (Prilosec) can be administered in the form of a packet, suspension, delayed release table or capsule, or an oral disintegrating tablet. In one embodiment, one dosage of omeprazole according to the subject composition is 2.5 mg to 40 mg, or 5 mg to 20 mg. In one embodiment, when administered in liquid form, the concentration of omeprazole is from 1 to 5 mg/ml, preferably 2 mg/ml per dose.

In certain embodiments, the supplement composition further comprises a biological amphiphilic molecule. In a specific embodiment, the biological amphiphilic molecule is a surfactant, preferably a biosurfactant. Biosurfactants are a structurally diverse group of surface-active substances produced by microorganisms. Biosurfactants are biodegradable and can be produced using selected organisms on renewable substrates. Most biosurfactant-producing organisms produce biosurfactants in response to the presence of a hydrocarbon source (e.g., oils, sugar, glycerol, etc.) in the growing media. Other media components such as concentration of iron can also affect biosurfactant production significantly.

Microbial biosurfactants are produced by a variety of microorganisms, such as, for example, Pseudomonas spp. (P. aeruginosa, P. putida, P. florescens, P. fragi, P. syringae); Flavobacterium spp.; Bacillus spp. (B. subtilis, B. pumillus, B. licheniformis, B. amyloliquefaciens, B. cereus); Wickerhamomyces spp. (e.g., W. anomalus), Candida spp. (e.g., C. albicans, C. rugosa, C. tropicalis, C. lipolytica, C. torulopsis); Rhodococcus spp.; Arthrobacter spp.; Campylobacter spp.; Cornybacterium spp.; Pichia spp. (e.g., P. anomala, P. guilliermondii, P. occidentalis); Starmerella spp. (e.g., S. bombicola); and so on.

All biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and change the properties of bacterial cell surfaces.

Biosurfactants accumulate at interfaces, thus reducing interfacial tension and leading to the formation of aggregated micellar structures. The ability of biosurfactants to form pores and destabilize biological membranes permits their use as antibacterial, antifungal, and hemolytic agents. Combined with the characteristics of low toxicity and biodegradability, biosurfactants are advantageous for use in a variety of application, including human health.

Biosurfactants include glycolipids, lipopeptides, flavolipids, phospholipids, fatty acid esters, and high molecular weight polymers such as lipoproteins, lipopolysaccharide-protein complexes, and polysaccharide-protein-fatty acid complexes.

The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), by an organic acid in the case of flavolipids, or, by a carbohydrate in the case of glycolipids.

In one embodiment, the biosurfactants according to the present invention are selected from glycolipids, such as rhamnolipids (RLP), sophorolipids (SLP), trehalose lipids (TL), cellobiose lipids and/or mannosylerythritol lipids (MEL).

In one embodiment, the biosurfactants are selected from lipopeptides, including, for example, surfactin, iturin, fengycin, arthrofactin, viscosin, amphisin and/or lichenysin.

In preferred embodiments, the composition comprises a glycolipid biosurfactant. In a specific embodiment, the glycolipid is a purified SLP. Certain SLP have the ability to destroy biofilms, and have anti-inflammatory, tissue-healing, antibacterial and/or antioxidant properties.

Sophorolipids are glycolipids that comprise a sophorose consisting of two glucose molecules, linked to a fatty acid by a glycosidic ether bond. They are categorized into two general forms: the lactonic form, where the carboxyl group in the fatty acid side chain and the sophorose moiety form a cyclic ester bond; and the acidic form, or linear form, where the ester bond is hydrolyzed. In addition to these forms, there exist a number of derivatives characterized by the presence or absence of double bonds in the fatty acid side chain, the length of the carbon chain, the position of the glycosidic ether bond, the presence or absence of acetyl groups introduced to the hydroxyl groups of the sugar moiety, and other structural parameters.

In preferred embodiments, the SLP according to the subject invention are represented by General Formula (1) and/or General Formula (2), and are obtained as a collection of 30 or more types of structural homologues having different fatty acid chain lengths (R³), and, in some instances, having an acetylation or protonation at R¹ and/or R².

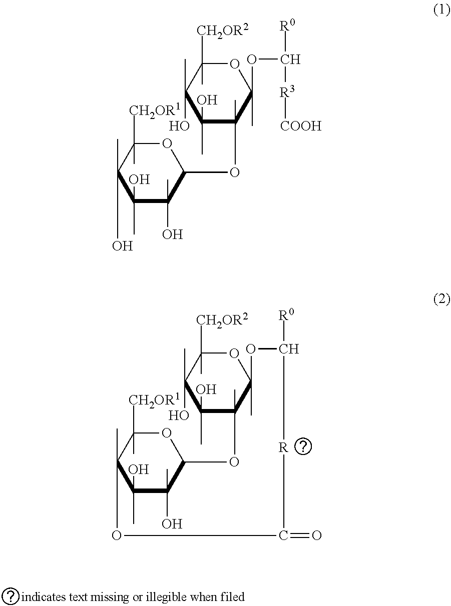

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more substituents.

Examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, and the like. R³ typically has 11 to 20 carbon atoms, preferably 13 to 17 carbon atoms, and more preferably 14 to 16 carbon atoms. Examples of the halogen atoms or halogen atoms bound to alkyl groups or alkoxy groups include fluorine, chlorine, bromine, and iodine.

In specific embodiments, the SLP is a lactonic form SLP according to (General Formula (2)). Lactonic SLP have greater antimicrobial and anti-biofilm capabilities that acidic SLP (General Formula (1)).

In certain embodiments, the concentration of purified SLP in the supplement composition is about 5 to 20 μg/ml, preferably about 7 to 4 μg/ml.

### Formulation and Delivery of Supplement Compositions

In one embodiment, the composition can be formulated for administering directly into the subject's GI tract. For example, the composition can be formulated for administration to the proximal lower GI via colonoscopy, the distal lower GI via enema or rectal tubes, and the upper GI tract via nasogastric tubes, duodenal tubes, and endoscopy/gastroscopy.

In one embodiment, the supplement composition is formulated so that it can be delivered to a subject orally. In particular, the composition is formulated as an orally-consumable product.

Orally-consumable products according to the invention are any preparations or compositions suitable for consumption, for nutrition, for oral hygiene or for pleasure, and are products intended to be introduced into the human or animal oral cavity, to remain there for a certain period of time and then to either be swallowed (e.g., food ready for consumption) or to be removed from the oral cavity again (e.g. chewing gums or products of oral hygiene or medical mouth washes). These products include all substances or products intended to be ingested by humans or animals in a processed, semi-processed or unprocessed state. This also includes substances that are added to orally-consumable products (e.g., active ingredients such as extracts, nutrients, supplements, or pharmaceutical products) during their production, treatment or processing and intended to be introduced into the human or animal oral cavity.

Orally-consumable products can also include substances intended to be swallowed by humans or animals and then digested in an unmodified, prepared or processed state. These include casings, coatings or other encapsulations that are intended also to be swallowed together with the product or for which swallowing is to be anticipated.

Preferably, the orally-consumable product according to the invention is formulated as a composition to be consumed for nutrition or pleasure. These particularly include baked goods (e.g., bread, dry biscuits, cake, cookies, brownies and other pastries), sweets and candies (e.g., chocolates, chocolate bar products, other bar products, gummies, fruit leathers, jelly beans, coated tablets, hard candies, toffees and caramels, and chewing gum), non-alcoholic beverages (e.g., cocoa, coffee, green tea, black tea, herbal teas, lemonades, isotonic beverages, soft drinks, nectars, fruit and vegetable juices, and fruit or vegetable juice preparations), instant beverages (e.g., instant, cocoa beverages, instant tea beverages, instant smoothies, instant milkshakes and instant coffee beverages), meat products (e.g., cold cuts, fresh or raw sausage preparations, seasoned oder, marinated fresh meat or salted meat products), eggs or egg products (e.g., dried whole egg, egg whites, and egg yolks), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, ice cream, sherbet, whey, butter, buttermilk, and partly or wholly hydrolyzed products containing milk proteins), products produced from nuts (e.g., nut milks, nut butters, nut flours or powders), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sorbets, fruit smoothies, fruit sauces, and fruit fillings), vegetable preparations (e.g., ketchup, sauces, dried vegetables, deep-freeze vegetables, pre-cooked vegetables, and boiled vegetables), snack articles (e.g., chips, crisps, pretzels, biscuits, crackers and nuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening beverages or other food). The present compositions may also serve as semi-finished products for the production of other compositions intended for nutrition or pleasure.

The composition of the subject invention can also be present in the form of capsules, tablets (uncoated and coated tablets, e.g., gastro-resistant coatings), coated tablets, granules, pellets, solid-substance mixtures, dispersions in liquid phases, as emulsions, powders, solutions, pastes or other swallowable or chewable preparations, or as a dietary supplement.

For oral administration, tablets or capsules can be prepared by conventional means with acceptable excipients such as binding agents, fillers, lubricants, disintegrants, or wetting agents. The tablets can be coated, if desired. Liquid preparations for oral administration can take the form of, for example, solutions, syrups, or suspension, or they can be presented as a dry product for constitution with saline or other suitable liquid vehicle before use.

In one embodiment, the supplement composition is formulated for delivery using a biosurfactant delivery system, wherein SLP form a liposome, or a micro- or nanocapsule, with the essential oils, and optionally the PPI, encapsulated therein. In one embodiment, additional biological polymers can be included to provide further structure for encapsulation.

Encapsulating can enhance the bioavailability of the supplement composition by protecting the encapsulated substances from components in the blood, such as proteins and other molecules, that otherwise might bind to the substances and prevent them from penetrating a target site. Additionally, the encapsulated delivery system can allow for antibacterial compounds that might otherwise be degraded by acids or enzymes in the GI tract to be administered orally, as it creates a barrier against the acids or enzymes. Furthermore, the encapsulated delivery system formulation allows for time release of the supplement composition, thereby reducing the potential toxicity or potential negative side-effects of a compound in a subject.

The compositions described herein can also contain acceptable additives as will be understood by one skilled in the art, depending on the particular form of the delivery method. Non-limiting examples of such additives include suspending agents, emulsifying agents, non-aqueous vehicles, preservatives, buffer salts, flavoring, coloring, and sweetening agents as appropriate. Non-limiting examples of specific additives include: gelatin, glycerin, water, beeswax, lecithin, cocoa, caramel, titanium dioxide, and carmine. Preparations for oral administration also can be suitably formulated to give controlled release of the active ingredients.

In some cases, the composition provided herein can contain an acceptable carrier for administration to a human subject or other mammal including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers appropriate to specific routes of administration.

Methods for Treating and/or Preventing H. pylori Infections

In preferred embodiments, the present invention provides methods for treating and/or preventing Helicobacter infection in a subject, wherein the method comprises administering to the subject a therapeutically-effective amount of a supplement composition of the present invention.

In some embodiments, the subject has been infected with Helicobacter. In some embodiments, the subject is at risk of being infected. Subjects who are at risk of being infected include, for example, subjects living in crowded living conditions, subjects living in unhygienic living conditions, subjects living without reliable clean water sources, subjects living in a developing country, and subjects living with someone who has been infected with Helicobacter.

In certain embodiments, the infection is caused by any pathogenic species of Helicobacter, including, but not limited to, H. bilis, H. bizzozeronii, H. canadensis, H. canis, H. cinaedi, H. fennelliae, H. heilmannii, H. hepaticus, H. pullorum, H. pylori, H. rappini, H. salmonis, and H. suis. In preferred embodiments, the infection is caused by H. pylori.

In certain embodiments, the methods comprise administering a therapeutically-effective amount of a blend of one or more essential oils selected from lemongrass oil, garlic oil, oregano oil, Satureja bachtiarica oil and ginger oil. In one embodiment, the essential oils are in the form of solutions, wherein the essential oil has been dissolved in an aqueous solvent, such as, e.g., distilled water.

In one embodiment, the method further comprises administering a therapeutically effective amount of a proton-pump inhibitor to the subject.

In one embodiment, the method further comprises administering a therapeutically effective amount of a biological amphiphilic molecule to the subject.

In one embodiment the method comprises administering a therapeutically effective amount of a composition that comprises, consists of, or consists essentially of a blend of therapeutically-effective amounts of the following essential oils: lemongrass oil, garlic oil, oregano oil, Satureja bachtiarica oil and ginger oil.

In one embodiment the method comprises administering a therapeutically effective amount of a composition that comprises, consists of, or consists essentially of a blend of therapeutically-effective amounts of the following essential oils: lemongrass oil, garlic oil, oregano oil, Satureja bachtiarica oil, ginger oil; a proton-pump inhibitor; and a biosurfactant.

In preferred embodiments, the PPI that is administered to the subject is omeprazole. Furthermore, in preferred embodiments, the biosurfactant that is administered to the subject is a glycolipid, even more preferably, SLP.

In one embodiment, the method further comprises testing the subject for and/or diagnosing the subject with H. pylori infection prior to administering a composition to the subject. In one embodiment, the testing is performed using known testing methods, including blood antibody test, urea breath test, stool antigen test, and stomach biopsy.

In one embodiment, the method further comprises performing follow-up tests on the subject to determine whether, and/or to what extent, the infection has been treated. The subject can be monitored throughout the course of treatment, for example, every day or every other day, in order to determine the status of the infection and whether or not the composition is effectively treating the infection. This can include, for example, performing tests, such as those used for diagnosing H. pylori infection, as well as observing the subject for signs of improving health. If follow-up tests show that the rate of improved health is below that which is desired, the dosage of the composition can be adjusted as determined by the skilled practitioner.

In certain embodiments, the present methods can be used to control H. pylori bacteria present in a subject's body, for example, in the GI tract. Additionally, in certain embodiments, the present methods can be used to treat and/or prevent symptoms, diseases, disorders and/or conditions that arise as a result of H. pylori infection. These can be symptoms, diseases, disorders and/or conditions of the digestive system, or they can afflict other parts of the body.

In one embodiment, the present invention can be used to treat and/or prevent digestive symptoms, diseases, disorders and/or conditions caused by H. pylori infection, including, but not limited to, stomach pain, nausea, vomiting, peptic ulcers, stomach cancer, gastritis, GI bleeding, diarrhea, constipation, gas, bloating, food sensitivities, heartburn, acid-reflux, GERD, and indigestion.

In some embodiments, the present invention can be used to treat and/or prevent extra-intestinal symptoms associated with a health condition affecting one or more body system, for example, the cardiovascular system, cerebrovascular system, nervous system, liver, or pancreas.

For example, in one embodiment, the subject is diagnosed with one or more of: atherosclerosis, coronary heart disease, acute ischemic stroke, and myocardial infarction, migraines, hepatocellular carcinoma, cirrhosis and hepatic encephalopathy, nonalcoholic fatty liver disease and fibrosis, acute and chronic pancreatitis pathogenesis, autoimmune pancreatitis, diabetes mellitus, metabolic syndrome, Alzheimer's disease, and Parkinson's disease, in addition to being infected with H. pylori.

In one embodiment, the present methods can be used to treat and/or prevent the occurrence of H. pylori-associated symptoms, diseases, disorders and/or conditions of the cardiovascular and/or cerebrovascular systems. H. pylori has been associated with cardiovascular and cerebrovascular diseases, such as atherosclerosis, coronary heart disease, acute ischemic stroke, and myocardial infarction, with the strains of bacteria capable of expressing the cytotoxin-associated gene A (Cag A) encoding the CagA protein, serving as the main cause. There are at least two hypothesized mechanisms regarding the bacteria's pathologic effects. First, H. pylori is thought to indirectly affect these systems by inducing systemic inflammation, which is a known risk factor of atherosclerosis. Bacterial infection localized in the GI tract induces dyslipidemia, hypercoagulability, and production of C-reactive proteins, increases the levels of fibrinogen, blood leukocyte and homocysteine, stimulates immune cross-reactivity, and initiates production of pro-inflammatory cytokines (e.g., interleukins, lymphocytes) and cytotoxic agents. This inflammation alters blood vessels motility, resulting in endothelial dysfunction and further, in plaque formation.

Second, H. pylori is thought to directly affect these systems through invasion of atherosclerotic and carotid plaques. H. pylori produces the toxins, vacuolating cytotoxin gen A (Vac A) and cytotoxin associated gene A (Cag A). Cag A is more virulent and participates in the formation of cholesterol patches and release of immune-mediated response through the release of cytokines, fibrinogen, triglycerides, high density lipoprotein, C-reactive protein, heat shock protein, and white blood cells. Cag A-producing strains also affect the activity of COX-1 and COX-2 in vascular endothelial cells. Furthermore, H. pylori infections may stimulate an inflammatory response against heat shock protein (HSP); therefore, an immune response to H. pylori may induce immune cross-reaction between human and bacterial HSP which in turns lead to an autoimmune reaction and local inflammation of the artery.

In one embodiment, the present methods can be used to treat and/or prevent the occurrence of H. pylori-associated migraines. When the immune system is activated by the pathogen, release of vasoactive substances is induced, as well as inflammatory responses, oxidative stress, nitric oxide imbalance and/or virulence of CagA-positive pathogens. Furthermore, the infection may affect the nervous system and aggregation of lipid peroxidation by-products in the bloodstream, which leads to migraine headaches.

In one embodiment, the present methods can be used to treat and/or prevent the occurrence of H. pylori-associated liver and/or pancreatic symptoms, diseases, disorders and/or conditions, such as, e.g., hepatocellular carcinoma, cirrhosis and hepatic encephalopathy, nonalcoholic fatty liver disease and fibrosis, acute and chronic pancreatitis pathogenesis, autoimmune pancreatitis, diabetes mellitus and metabolic syndrome. H. pylori is able to adhere to and penetrate human hepatocytes, where it is hypothesized to affect gene expression of interleukin-8 (IL-8) and transforming growth factor-beta 1 (TGF-β1). At the same time, the bacterial release of toxins, such as ammonia and lipopolysaccharides (LPS), as well as the induction of inflammatory cytokines, may damage the pancreas tissue.

In one embodiment, the present methods can be used to treat and/or prevent the occurrence of H. pylori-associated neural and/or cognitive symptoms, diseases, disorders and/or conditions. Alzheimer's disease infected with H. pylori the level of cognitive impairment is more severe than in those, in whom H.pylori was not identified. It was assumed, that H. pylori strains, which express CagA enhances the existent neuroinflammation, inducing the IL-8 and TNF-alfa production. As well, the role of H. pylori was proposed to take place in Parkinson's disease.

Advantageously, the present invention can lead to simultaneous improvement of diseases, disorders and conditions caused by H. pylori infection, reduction in the occurrence of H. pylori infections, and reduction in the development of antibiotic-resistant strains of the bacteria.

In embodiments of the present invention, administration of the supplement composition occurs daily for several days or longer. Administration can include any known method of drug administration, including, but not limited to, oral, nasal, cutaneous (e.g., applying it as a cream), or intravenous administration.

In one embodiment, the supplement composition is ingested by the subject once, twice, or three times per day, determined on a subject-by-subject basis by a skilled physician. Factors to be considered when determining the number of doses to administer include the age of the individual receiving treatment and the severity of the subject's symptoms.

### REFERENCES

Ansari B, Bashi K, Meamar R, Chitsaz A , Nematollahi S. (2015). Association of Helicobacter pylori antibodies and severity of migraine attack. Iran J Neurol 2015; 14(3): 125-129. (“Ansari et al. 2015”).

Corrado G, Luzzi I, Lucarelli S. (1998). Positive Association between Helicobacter pylori Infection and Food Allergy in Children. Scandinavian Journal of Gastroenterology, 33(11), 1135-1139. doi:10.1080/00365529850172467. (“Corrado et al. 1998”).

Hosseinzadeh M, Khosravi A, Saki K, Ranjbar R. (2011). Evaluation of Helicobacter pylori infection in patients with common migraine headache. Arch Med Sci 2011; 7(5): 844-9. (“Hosseinzadeh et al. 2011”).

Roubaud-Baudron, C., Krolak-Salmon, P., Quadrio, I., Mégraud, F., & Salles, N. (2012). Impact of chronic Helicobacter pylori infection on Alzheimer's disease: preliminary results. Neurobiology of Aging, 33(5), 1009.e11-1009.e19. doi:10.1016/j.neurobiolaging.2011.10.02. (“Roubaud-Baudron et al. 2012”).

1. A supplement composition comprising the following essential oils:
lemongrass oil,
garlic oil,
oregano oil,
Satureja bachtiarica oil, and
ginger oil,
wherein the essential oils are present in sufficient concentrations for the supplement composition to be therapeutically effective.

2. The composition of claim 1, further comprising a proton-pump inhibitor (PPI).

3. The composition of claim 2, wherein the PPI is omeprazole.

4. The composition of claim 1, further comprising a biosurfactant.

5. The composition of claim 4, wherein the biosurfactant is a sophorolipid (SLP).

6. A supplement composition consisting essentially of an essential oil blend comprising lemongrass oil, garlic oil, oregano oil, Satureja bachtiarica oil, and ginger oil,
a PPI, and
a biosurfactant.

7. The composition of claim 6, wherein the PPI is omeprazole.

8. The composition of claim 6, wherein the biosurfactant is a sophorolipid (SLP).

9-10. (canceled)

11. A method for treating and/or preventing Helicobacter infection in a subject, wherein the method comprises administering to the subject a therapeutically-effective amount of the composition of claim 1.

12. The method of claim 11, further comprising administering a PPI to the subject.

13. The method of claim 12, wherein the PPI is omeprazole.

14. The method of claim 11, further comprising administering a biosurfactant to the subject.

15. The method of claim 14, wherein the biosurfactant is a purified sophorolipid (SLP).

16. The method of claim 11, wherein the supplement composition is administered to the subject orally.

17. The method of claim 11, further comprising diagnosing the subject with an Helicobater infection prior to administering the supplement composition.

18. The method of claim 17, wherein the infection is caused by one or more of H. bilis, H. bizzozeronii, H. canadensis, H. canis, H. cinaedi, H. fennelliae, H. heilmannii, H. hepaticus, H. pullorum, H. pylori, H. rappini, H. salmonis, and H. suis.

19. The method of claim 18, wherein the infection is caused by H. pylori.

20. The method of claim 11, further comprising performing follow-up tests on the subject to determine whether, and/or to what extent, the infection has been treated,

21. The method of claim 11, used to treat and/or prevent a symptom, disease, disorder or condition of the digestive tract caused by Helicobacter infection, wherein the symptom, disease, disorder or condition is selected from stomach pain, nausea, vomiting, peptic ulcers, stomach cancer, gastritis, GI bleeding, diarrhea, constipation, gas, bloating, food sensitivities, heartburn, acid-reflux, GERD, and indigestion.

22. (canceled)

23. The method of claim 11, used to treat and/or prevent extra-intestinal symptoms associated with a health condition affecting one or more of the cardiovascular system, cerebrovascular system, nervous system, liver, and pancreas.
